(19) United States
(12) Patent Application Publication
(10) Pub. No.: US 2026/0335039 A1
(43) Pub. Date: Oct. 29, 2026

(54) Method and Apparatus For Sleeved Loupes

(71) Applicant: Lumadent Corporation, Reno, NV (US)

(72) Inventor: Duong Ton, Reno, NV (US)

(21) Appl. No.: 19/240,337

(22) Filed: Jun. 17, 2025

Related U.S. Application Data
(60) Provisional application No. 63/795,531, filed on Apr. 27, 2025.

(51) Int. Cl. A61B 90/35; A61B 90/30; A61B 90/50

(52) U.S. Cl. CPC A61B 90/35; A61B 2090/309; A61B 2090/502

(57) ABSTRACT

A modular loupe apparatus includes a wearable frame, a sleeve supported relative to the frame, a removable loupe assembly sized to be received in the sleeve, and a locking mechanism positioned in the sleeve to hold the loupe assembly during use and allow removal of the loupe assembly. In some implementations, the sleeve is supported by a mono-post crane apparatus mounted to an eyeglass-frame top bar and coupled to a horizontal suspension bar by a tie.

### INCORPORATION BY REFERENCE

This application is a non-provisional application of and claims the benefit of earlier filing date under 35 U.S.C. 119(e) to the filing date of U.S. Application No. 63/795,531, filed on 2025 Apr. 27, titled “Method and Apparatus for Loupes with Sleeve,” the entirety of which is incorporated herein by reference.

### FIELD OF INVENTION

The present invention relates generally to magnification systems used in dentistry and surgery, and more particularly to an apparatus and method for modular loupes utilizing a sleeve-based mounting system to enable interchangeable loupe components for improved customization, weight management, and ergonomic use.

### BACKGROUND

Dental and surgical loupes are essential tools that provide magnification to improve the precision of medical and dental procedures. Conventional loupes are typically permanently affixed to eyeglass frames or headbands and configured with fixed optical parameters such as magnification power, working distance, and focal alignment. While such systems can be customized for individual users, they lack the flexibility to adapt to changing needs during different procedures or across multiple users.

For instance, a dentist may require a different magnification level for general cleaning versus endodontic surgery, yet current loupe systems necessitate changing the entire eyeglass setup to accommodate this. Additionally, fixed-mount loupes can cause strain due to their weight and poor balance, especially when higher magnification is used. Furthermore, maintenance and sterilization can be cumbersome if the optical components cannot be detached easily.

There remains a need for a loupe system that, while maintaining personalized fit and alignment, light weight, and ergonomic comfort, enables the use of interchangeable magnifying optics. The present invention addresses these needs. It advances novel loupes with sleeve such that different magnification or specialty loupes can be swappable. The swapable loupes have minimal weight differences, and the frame and glasses can be personalized for each medical professionals.

### OBJECTIVE OF THE INVENTION

It is an object of this invention to provide an apparatus of sleeved loupes that are interchangeable for dentistry and medical uses.

It is an object of this invention to provide an apparatus of sleeved loupes that are easily interchangeable with a spring-loaded latch.

It is an object of this invention to provide an apparatus of sleeved loupes that are easily interchangeable with a built-in magnet.

It is also an object of this invention to disclose a method to create an apparatus of a system of interchangeable sleeved loupes.

### SUMMARY OF THE INVENTION

This invention is directed to a modular loupe apparatus that comprises a frame configured to be worn by a user, a sleeve fixedly attached to the frame, a loupe assembly configured to be removably inserted into the sleeve, and a locking mechanism disposed within the sleeve, wherein the locking mechanism secures the loupe assembly during use and permits removal thereof.

In another embodiment, the locking mechanism comprises a spring-loaded latch.

In yet another embodiment, the locking mechanism comprises a magnetic coupling.

In another embodiment, the apparatus further comprises alignment features within the sleeve to guide the loupe assembly into a predetermined orientation.

In another embodiment, the loupe assembly includes optical elements configured to provide a magnification between 2.0× and 6.0×.

In another embodiment, the sleeve is made of a sterilizable polymer material.

In another embodiment, the apparatus further comprises a release tab accessible from an exterior of the sleeve for disengaging the locking mechanism.

In another embodiment, the frame can be an eyeglass frame.

In another embodiment, the frame can be a headband.

In another embodiment, the loupe assembly includes one or more light-emitting diodes (LEDs).

In another embodiment, the sleeve includes electrical connectors for powering the one or more LEDs from a battery source.

This invention is further directed to a method for a modular loupe system that comprises providing a frame configured to be worn by a user, providing a sleeve, attaching the sleeve to the frame, providing a loupe assembly, inserting the loupe assembly into the sleeve, providing a locking mechanism, engaging the locking mechanism to secure the loupe assembly to the sleeve when in use, and removing the loupe assembly by disengaging the locking mechanism.

In another embodiment, the engaging the locking mechanism step comprises inserting the loupe assembly until a spring-loaded latch is actuated.

In another embodiment, the engaging the locking mechanism step comprises aligning embedded magnets disposed in the sleeve and the loupe assembly.

In another embodiment, the method further comprises illuminating a surgical field with an LED integrated into the loupe assembly.

### BRIEF DESCRIPTION

The present invention will be understood and appreciated more fully from the following detailed description taken in conjunction with the drawings in which:

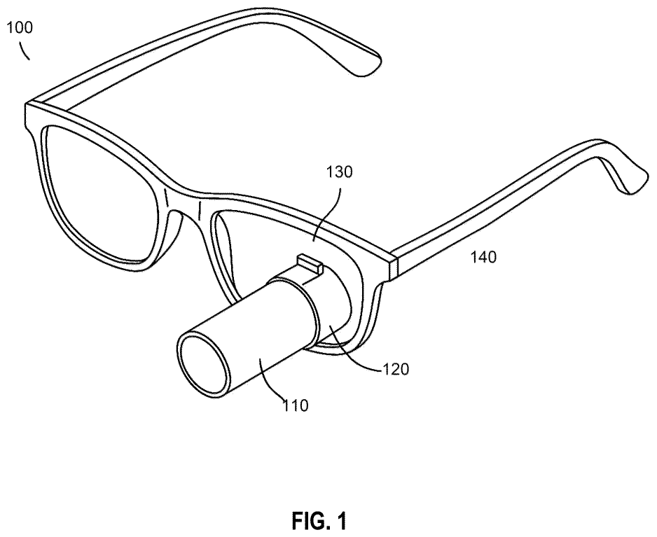

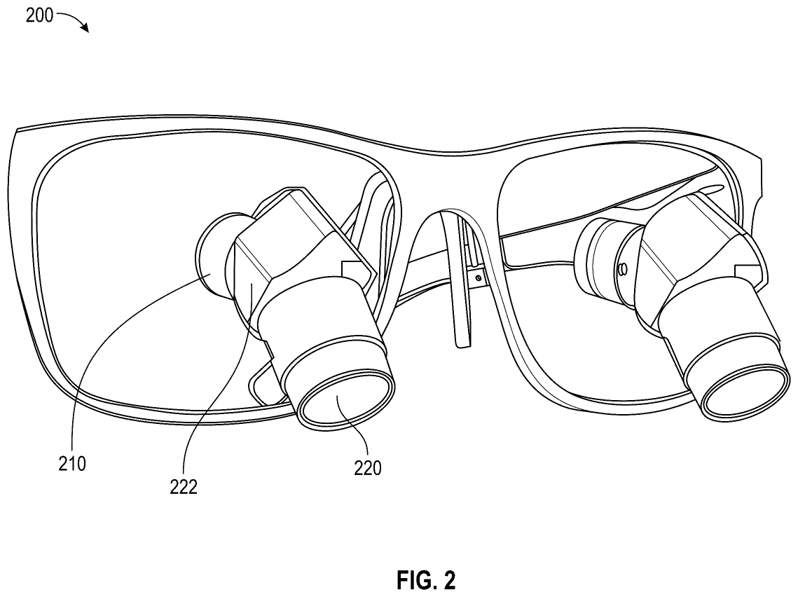

FIG. 1 is a perspective view of a modular loupe apparatus with sleeve and detachable loupe affixed to a carrier eyeglass.

FIG. 2 is a perspective view of another embodiment of the present invention where the sleeved loupe apparatus incorporates a prism to give it a down angle view.

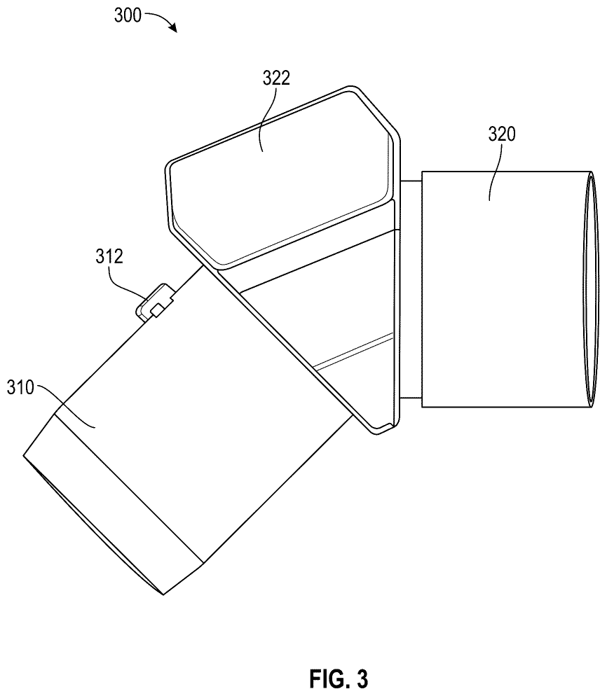

FIG. 3 is a side view of another embodiment of the present invention, similar to the embodiment disclosed in FIG. 2, where the sleeved loupe apparatus incorporates a pin bolt to secure the detachable lens inside the sleeve.

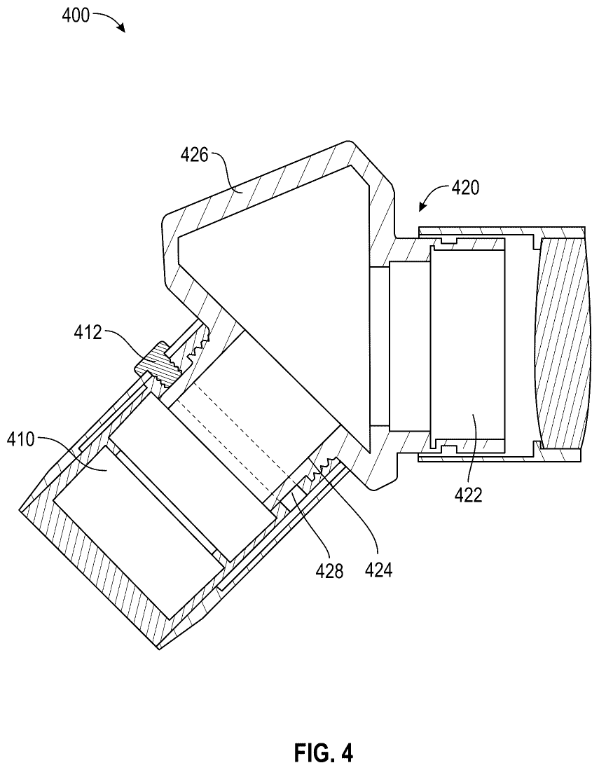

FIG. 4 is a cross-sectional view of an embodiment of the present invention showing how a pin bolt secures the lens inside the sleeve.

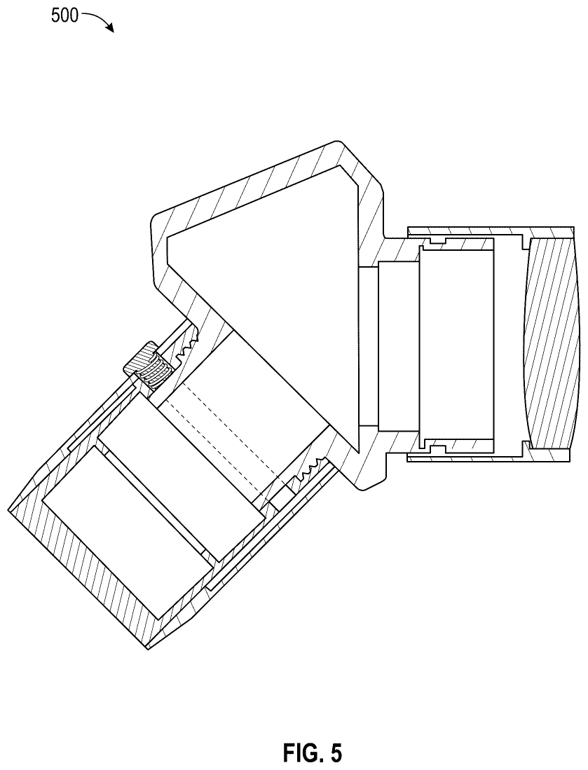

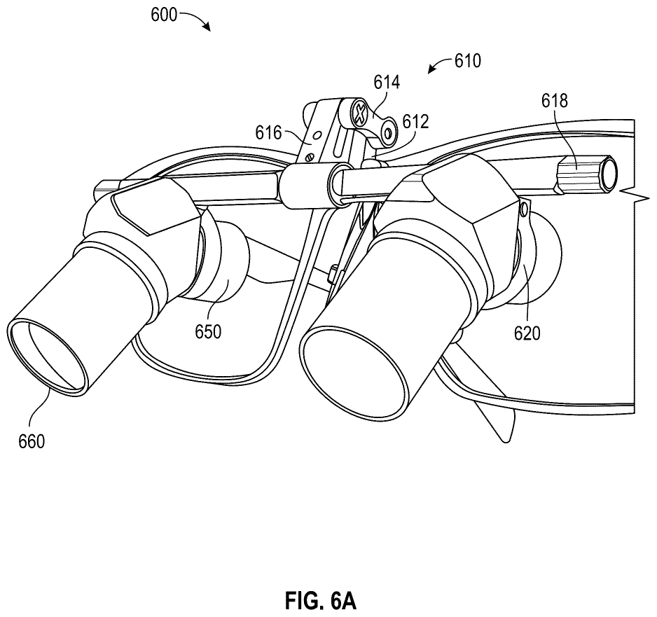

FIG. 5 is a cross-sectional view of another embodiment of the present invention showing how the pin that secures the lens inside the sleeve is attached to a spring that is disposed inside the sleeve to pull and press the pin against the sleeve but allow it to move up and down within a limited distance when a user applies a force.

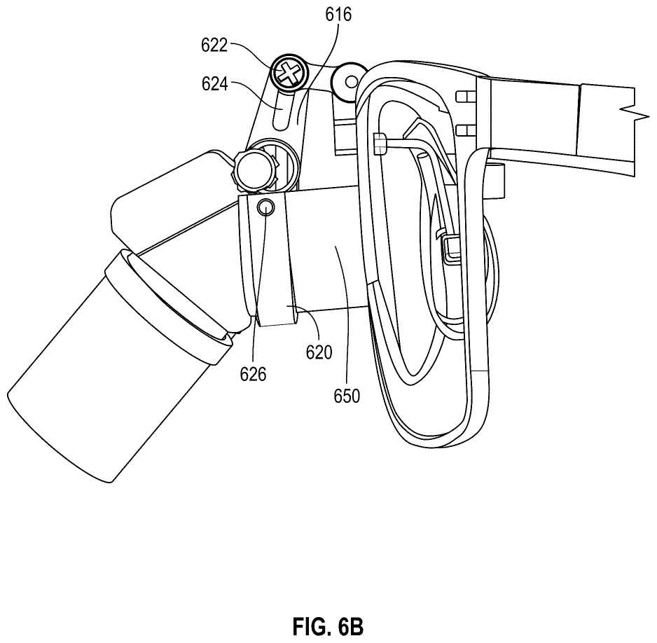

FIG. 6A is a perspective view of another embodiment of the present invention showing a sleeved loupe apparatus that is suspended in front of the carrier glass by a monopost crane apparatus that is attached to the crossbar of the eyeglass frame.

FIG. 6B is a side view of the embodiment of the present invention disclosed in FIG. 6A.

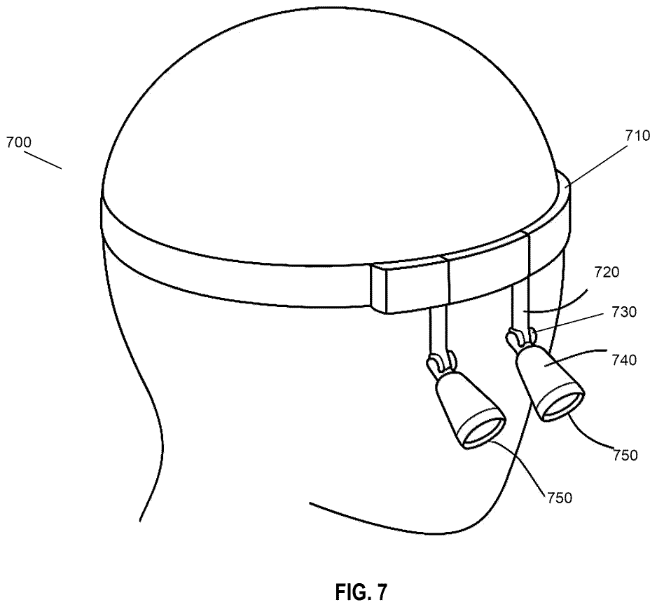

FIG. 7 shows another embodiment of the present invention that comprises a headband-mounted variant with two interchangeable sleeved loupes.

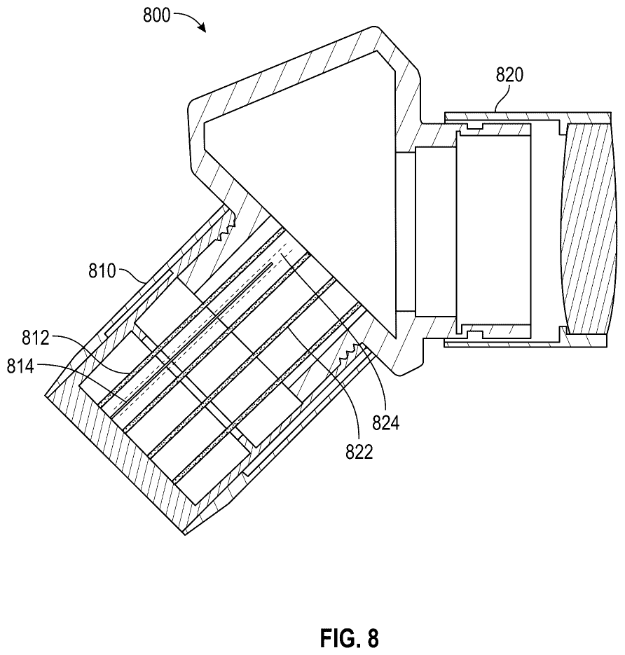

FIG. 8 is a cross-sectional view of an alternative embodiment of the present invention that utilizes magnetic coupling between the sleeve and loupe to secure the loupe inside the sleeve.

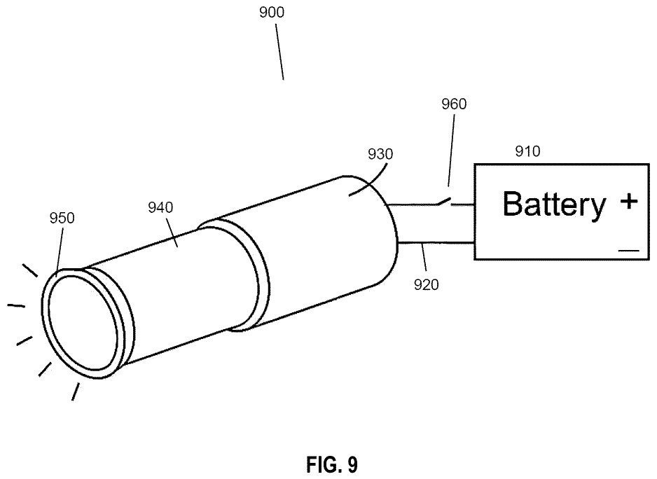

FIG. 9 depicts another embodiment of the present invention that incorporates an integrated LED lighting and electrical power source to illuminate a surgical field.

### DETAILED DESCRIPTION

The invention will be described in the context of a preferred embodiment.

Referring now to the figures, FIG. 1 discloses an exemplary embodiment 100 of the present invention. This embodiment is a modular loupe system 100 in which a loupe 110 is mounted to a sleeve 120 which in turn is mounted to a carrier glass 130. Glass 130 is inserted into a frame 140 like a pair of traditional reading glasses. Sleeve 120 is permanently affixed to the carrier lens 130 and forms a cylindrical receptacle for the loupe's 110 barrel. Loupe 110 contains one or more optical lenses configured to provide a specific magnification suitable for medical or dental procedures.

FIG. 2 discloses another embodiment 200 of the present invention that is similar to the embodiment disclosed in FIG. 1 in that the sleeve 210 is mounted to a carrier glass, but the loupe 220 incorporates a prism 222 to have the loupe bent down at an angle so that the medical professional does not have to bend his or her neck too much to look at the surgical field for a period of time during a procedure. Thus, this embodiment improves the ergonomics of the present invention.

FIG. 3 shows another embodiment 300 of the present invention that comprises a sleeve 310 that in turn comprises a latch pin 312. Embodiment 300 further comprises a loupe 320 that in turn comprises a prism 322 to allow the loupe to form an angle with the axis of the sleeve. A part of the loupe is inserted into the sleeve and locked in place by the latch pin 322. This arrangement of the embodiment 300 allows the loupe to be detachable and this sleeved loupe apparatus is interchangeable for different magnification.

FIG. 4 shows a cross-sectional illustration of an exemplary embodiment 400 of the present invention that similarly comprises a sleeve 410 that in turn comprises a latch pin 412. Embodiment 400 further comprises a loupe 420 that in turn comprises two halves, an objective lens 422 that faces the surgical field, and an eyepiece half 424 that is inserted into the sleeve 410. The two halves are connected to a prism 426 in the middle to give the loupe an angle which improves the ergonomics of this sleeved loupe apparatus. The eyepiece half 424 further comprises a groove 428 on the outside of its barrel. To insert the eyepiece half into the sleeve, the user momentarily lifts the latch pin 412 to allow the eyepiece half to slide in, and, then, pushes or screw the latch pin down so it can engage with the groove 428 and prevent the eyepiece half and the loupe from dropping out of the sleeve. To detach the loupe from the sleeve, the user lifts the latch pin from the groove so that the loupe can be slid out of the sleeve.

FIG. 5 shows a variant of Embodiment 400. This embodiment 500 comprises a latch pin 510 that is spring-loaded to facilitate easy attachment and detachment of the loupe. The spring 520 is disposed inside the sleeve and presses against the inner wall of the sleeve's barrel. In its rest state, the spring 520 pushes the latch pin down in a groove 530 on the eyepiece half of the loupe. When the user needs to attach or detach the loupe from the sleeve, the user just lifts the latch pin to free the loupe from its locked position. When the user wants to insert a different loupe, the user just lifts the latch pin up so that the loupe's barrel can be slid in the sleeve's barrel and, then, lets go of the latch pin to return it to its rest state so it engages with the groove 530 and prevents the loupe from dropping out of the sleeve.

FIG. 6A discloses another embodiment 600 of the present invention that, instead of having the sleeve affixed to the carrier lens, comprises a mono-post crane apparatus 610 that is mounted on and hung off the eyeglass frame's top bar and a sleeve 650 is attached to the crane apparatus by a metal tie 620. This crane apparatus eliminates the need for the sleeve to be permanently affixed to the carrier lens. The crane apparatus 610 further comprises a bracket 612 that is attached to the eyeglass frame's top bar and a first extension arm 614 attached to the bracket with a bolt at its one end that allows the extension arm to swivel up and down and toward the front of the eyeglass frame. Apparatus 610 further comprises a second extension arm 616 attached to the first extension arm with a bolt at its one end. The second extension arm can also swivel relative to the first extension arm, and when in use, it is positioned in the front of the eyeglass frame and pointing down so that other parts and the sleeved loupe can hang on it and be at the user's eye level. The second arm is adjustable to the user's eye level. To achieve this utility, it has an elongated cutout or slot running its length that allows the joint bolt to slide in it. Apparatus 610 further comprises horizontal suspension bar 618 attached to the other end of the second arm 616. The suspension bar extends left and right from the center of the eyeglass frame to about the user's left and right eye. The left and right extensions of the suspension bar allow a left and right loupe to be attached and suspended on it. Apparatus 610 further comprises a metal tie 620 that is attached to the suspension bar and ties the sleeve 650 of the sleeved loupe to the suspension bar. It is appreciated that the tie could be made of rubber or other materials suitable for a strong tie. Once the whole suspension apparatus is set up and adjusted for the user's eye level, the user can easily exchange a loupe 660 for another loupe with a different magnification.

FIG. 6B shows a side view of Embodiment 600 disclosed in FIG. 6A and further discloses the embodiment in detail. As disclosed above, the second arm 616 comprises a slot 624 that allows the joint bolt 622 to go through and be disposed in and through the center of the slot. This design allows the second arm and the suspension bar attached to it to be adjusted up and down relative to the eyeglass frame and the user's eyes so as the sleeved loupe attached to it. Furthermore, the tie 620 comprises a bolt 626 that allows the tie to be tightened such that the sleeve 650 is securely attached to the suspension apparatus.

FIG. 7 discloses another embodiment 700 of the present invention that comprises a padded headband 710 that in turn comprises bridges 720 onto which modular sleeves 740 are mounted by a pivot bolt 730. Loupes 750 are mounted on the modular sleeves 740 with a built-in latch mechanism. Each sleeve 740 accepts mating loupe barrels that are held inside the sleeves with the built-in latch. The interchangeable loupes can have different and distinct magnifications. The headband configuration allows for weight redistribution away from the nose and temples, enhancing user comfort during prolonged procedures. The pivot bolt 730 allows the sleeves 740 to swivel and change its radial direction relative to the bridge 720 and the headband 710 and, therefore, the user can change the view angle to align with his or her sight.

FIG. 8 discloses another embodiment 800 of the present invention that employs magnetic coupling to hold the loupes inside the sleeves. Embodiment 800 comprises a sleeve 810 and a loupe 820. Both sleeve 810 and loupe 820 comprise embedded magnets 812 and 822, respectively, and these magnet strips are embedded in the sleeve and loupe barrels and run the lengths of both. For Sleeve 810, the magnet strips 812 are disposed flush or close to the inner surface of its barrel. For Loupe 820, the magnet stripes 822 are disposed flush or close to the outer surface of its barrel. In a variant embodiment, to align the magnet strips, there are additional mating grooves 824 and ridges 814 to align the magnet strips and other components of the sleeve and loupe. These additional grooves and ridges and/or magnetic forces will align the optical elements, if required, and secure the loupe inside the sleeve without mechanical components such as latch pins.

FIG. 9 discloses another embodiment 900 of the present invention that comprises a battery source 910 that provides the needed electricity to light up the surgical field in front of the user. Embodiment 900 further comprises a pair of positive and negative electrical lines, which can be in a variety of forms, such as wires, plugs, pins, prongs, or blades. Like conventional electrical connectors, there is a mating receiver disposed in the body of the sleeve 930. When all the connectors are connected, the battery source 910 provides an electrical current for the lighting elements of the sleeved loupe apparatus of the present invention. The sleeve 930 comprises a female socket that is connected to the battery 910 on the back end and faces the loupe 940 on the front end. To run the electrical current to the integrated lighting element 950 in front of the loupe 940, this loupe comprises male protruding contacts that mate with the female socket of the sleeve 930 when the loupe is inserted in the sleeve. This embodiment further comprises an electrical switch 960 to turn the light on and off. The battery source 910 is mounted on the headband or frame of the present innovative apparatus. The lighting elements and this embodiment provide improved visibility in surgical fields without the need for external lighting arms that could clutter the surgical space.

All embodiments are constructed using sterilizable, lightweight materials such as anodized aluminum, medical-grade polymers, or carbon-reinforced composites. The apparatus allows practitioners to adapt optical configurations quickly and efficiently without compromising alignment or comfort.

1) A modular loupe apparatus comprising:
a frame configured to be worn by a user;
a frame-supported sleeve supported by said frame, said frame-supported sleeve defining a receptacle and remaining supported by said frame when an interchangeable loupe assembly is removed from said receptacle;
an interchangeable loupe assembly comprising a loupe barrel and one or more optical lenses, said loupe barrel being removably receivable in said receptacle of said frame-supported sleeve;
wherein said frame-supported sleeve is configured to receive different interchangeable loupe assemblies without replacing said frame; and
a locking mechanism disposed at said frame-supported sleeve and configured to engage said loupe barrel to secure said interchangeable loupe assembly in said frame-supported sleeve during use and permit removal of said interchangeable loupe assembly from said frame-supported sleeve.

2) The apparatus of claim 1, wherein said locking mechanism comprises a spring-loaded latch, a magnetic coupling, or both.

3) (canceled)

4) The apparatus of claim 1, further comprising alignment features within said frame-supported sleeve to guide said interchangeable loupe assembly into a predetermined orientation.

5) The apparatus of claim 1, wherein said interchangeable loupe assembly includes optical elements configured to provide a magnification between 2.0× and 6.0×.

6) The apparatus of claim 1, wherein said frame-supported sleeve is made of a sterilizable polymer material.

7) The apparatus of claim 1, further comprising a release tab accessible on an exterior of said frame-supported sleeve for disengaging said locking mechanism.

8) The apparatus of claim 1, wherein said frame comprises an eyeglass frame having a carrier lens, and wherein said frame-supported sleeve is fixedly attached to said carrier lens.

9) The apparatus of claim 1, wherein said frame comprises a padded headband comprising a bridge, and wherein said frame-supported sleeve is mounted to said bridge by a pivot bolt configured to permit said frame-supported sleeve to swivel relative to said padded headband.

10) The apparatus of claim 1, wherein said interchangeable loupe assembly includes one or more light-emitting diodes (LEDs).

11) The apparatus of claim 10, wherein said frame-supported sleeve comprises a female socket electrically connected to a battery source, and wherein said interchangeable loupe assembly comprises male protruding contacts configured to mate with said female socket when said interchangeable loupe assembly is inserted into said frame-supported sleeve to power said one or more LEDs.

12) A method for a modular loupe system, comprising:
providing a frame configured to be worn by a user;
providing a sleeve supported by said frame and defining a receptacle, said sleeve remaining supported by said frame when an interchangeable loupe assembly is removed from said receptacle;
providing an interchangeable loupe assembly comprising a loupe barrel and one or more optical Lenses;
inserting said loupe barrel into said receptacle of said sleeve;
engaging a locking mechanism disposed at said sleeve with said loupe barrel to secure said interchangeable loupe assembly to said sleeve during use; and
removing said interchangeable loupe assembly from said sleeve by disengaging said locking mechanism while said sleeve remains supported by said frame.

13) The method of claim 12, wherein engaging said locking mechanism comprises allowing a spring-loaded latch pin to engage a groove on said loupe barrel after said loupe barrel is inserted into said receptacle.

14) The method of claim 12, wherein engaging said locking mechanism comprises aligning first magnet strips embedded in said sleeve with second magnet strips embedded in said loupe barrel, said first magnet strips and said second magnet strips running along lengths of said sleeve and said loupe barrel, respectively.

15) The method of claim 12, further comprising illuminating a surgical field with an LED integrated into said interchangeable loupe assembly by supplying electrical power from a battery source to said LED through a female socket of said sleeve and male protruding contacts of said interchangeable loupe assembly.

16) The apparatus of claim 1, wherein said interchangeable loupe assembly comprises a prism connecting an objective lens portion and an eyepiece portion of said interchangeable loupe assembly.

17) The apparatus of claim 2, wherein said spring-loaded latch comprises a latch pin and a spring disposed inside said frame-supported sleeve, wherein said loupe barrel comprises a groove, and wherein said spring biases said latch pin into said groove to secure said interchangeable loupe assembly within said frame-supported sleeve.

18) The apparatus of claim 2, wherein said magnetic coupling comprises first magnet strips embedded in said frame-supported sleeve and second magnet strips embedded in said loupe barrel, wherein said first magnet strips and said second magnet strips run along lengths of said frame-supported sleeve and said loupe barrel, respectively.

19) The apparatus of claim 18, wherein said frame-supported sleeve and said loupe barrel comprise mating ridges and grooves configured to align said first magnet strips and said second magnet strips.

20) The apparatus of claim 1, wherein said frame comprises an eyeglass frame having a top bar, and wherein the modular loupe apparatus further comprises a mono-post crane apparatus mounted to said top bar, said mono-post crane apparatus comprising a bracket attached to said top bar, a first extension arm attached to said bracket, a second extension arm attached to said first extension arm, and a horizontal suspension bar attached to said second extension arm, wherein said frame-supported sleeve is attached to said horizontal suspension bar by a tie.

21) The apparatus of claim 20, wherein said second extension arm comprises an elongated slot configured to receive a joint bolt to adjust said second extension arm relative to said eyeglass frame, wherein said tie comprises a bolt configured to tighten said tie around said frame-supported sleeve, wherein said frame-supported sleeve is a first frame-supported sleeve, and wherein the modular loupe apparatus further comprises a second frame-supported sleeve supported by said horizontal suspension bar, said first frame-supported sleeve and said second frame-supported sleeve corresponding to a left eye and a right eye of said user.
